(19) United States
(12) Patent Application Publication
(10) Pub. No.: US 2014/0251088 A1
(43) Pub. Date: Sep. 11, 2014

(54) TATTOO MACHINE

(71) Applicant: EGOINK LIMITED, Sunderland (GB)

(72) Inventor: Richard . BESTON, Sunderland (GB)

(73) Assignee: Egoink Limited, Sunderland (GB)

(21) Appl. No.: 13/789,079

(22) Filed: Mar. 7, 2013

(51) Int. Cl. A61M 37/00

(52) U.S. Cl. CPC A61M 37/0076
USPC 81/9.22; 606/186

(57) ABSTRACT

A tattoo machine needle bar holder includes a housing configured to receive a needle bar therein; a first buffer element; and a second buffer element. The first and second buffer elements are adapted to be located within the housing, and in use, at least partially surround a portion of a needle bar located within the housing. The first buffer element is also adapted to provide a first dampening effect on the lateral transition of a needle bar located within the housing in a first direction, and the second buffer element is also adapted to provide a second dampening effect on the lateral transition of the needle bar in a second direction.

### FIELD OF INVENTION

The present invention relates to an improved tattoo machine.

The present invention also relates to a tattoo machine assembly and a tattoo machine kit.

### BACKGROUND

Tattooing is the act or practice of marking the skin with indelible patterns, pictures, legends, etc., by making punctures in it and inserting pigments.

This can be accomplished by hand, by use of a machine, or any other suitable method for the insertion of ink into the dermis layer of the skin to change the pigment.

Modern tattoo machines come in a number of different forms. They can vary from both a mechanical stand point and a technical stand point.

From a mechanical stand point, modern tattoo machines can be in the form of a coil tattoo machine, a pneumatic tattoo machine, or a rotatory or linear tattoo machine.

Coil tattoo machines use an electromagnetic circuit to move the needle. There are many variations, from single-coiled machines to triple-coiled machines.

A pneumatic tattoo machine is powered by an air compressor, and is generally extremely lightweight. Pneumatic tattoo machines use pressurized air to power the tattoo machine and drive the needles up and down.

Rotary or linear type machines use an electric motor to drive the needles.

From a technical stand point, a tattoo machine can be classed as a liner tattoo machine or a shader tattoo machine.

Liner tattoo machines are adapted to lay the ink in the skin in one single pass to create a dominant line. It uses a short contact circuit (about 1.5 mm-2 mm), which causes the machine to cycle faster.

Shader tattoo machines are commonly used to shade black or variants of black ink and to add color. It uses a bigger contact gap than a liner machine (about 2 mm-3.5 mm) to make it cycle slightly slower.

Most modern tattoo machines can control needle depth, speed, and force of application, which allows tattooing to become a very precise art form.

Tattoo artists are very skilled in their trade and will generally need to tweak or modify a machine to meet their particular preference. In addition, a plurality of machines may be required in order to be able to perform a wide range of tattooing techniques.

### SUMMARY OF INVENTION

In a first aspect, the present invention provides a tattoo machine needle bar holder comprising
a housing configured to receive a needle bar therein;
a first buffer element; and
a second buffer element;
   wherein the first and second buffer elements are adapted to be located within said housing, and in use, at least partially surround a portion of a needle bar located within said housing,
   wherein the first buffer element is adapted to provide a first dampening effect on the lateral transition of a needle bar located within said housing in a first direction,
   and the second buffer element is adapted to provide a second dampening effect on the lateral transition of the needle bar in a second direction.

By means of the invention, the effect of front and back springs of a coil tattoo machine can simulated with a rotary or linear tattoo machine.

Optionally, the first buffer element comprises a resilient material.

Optionally, the resilient material comprises a rubber material.

Optionally, the rubber material comprises an IRHD hardness value of between 10 and 100.

Optionally, the second buffer element comprises a resilient material.

Optionally, the resilient material comprises a rubber material.

Optionally, the rubber material comprises an IRHD hardness value of between 10 and 100.

Optionally, the first buffer element is removably locatable in the housing.

Optionally, the second buffer element is removably locatable in the housing.

Optionally, the first dampening effect is the same as the second dampening effect.

Optionally, the first dampening effect is different to the second dampening effect.

Optionally, the first dampening effect is different to the second dampening effect.

Optionally, the first dampening effect is greater than the second dampening effect.

Optionally, the first dampening effect is less than the second dampening effect.

Optionally, the first direction is opposite the second direction.

In accordance with a second aspect, there is provided a tattoo machine comprising:
a needle bar configured to translate in a lateral direction;
a motor adapted to actuate said needle bar in a lateral direction;
a tattoo needle tube holder; and
a housing configured to receive the needle bar therein;
a first buffer element; and
a second buffer element;
   wherein the first and second buffer elements are adapted to be located within said housing, and in use, at least partially surround a portion of the needle bar,
   wherein the first buffer element is adapted to provide a first dampening effect on the lateral transition of the needle bar in a first direction, and the second buffer element is adapted to provide a second dampening effect on the lateral transition of the needle bar in a second direction.

In accordance with a third aspect, there is provided a tattoo machine assembly comprising:
a needle bar configured to translate in a lateral direction;
a motor adapted to actuate said needle bar in a lateral direction;
a tattoo needle tube holder;
a tattoo needle tube;
a housing configured to receive the needle bar therein;
a first buffer element; and
a second buffer element;
   wherein the first and second buffer elements are adapted to be located within said housing, and in use, at least partially surround a portion of the needle bar,
   wherein the first buffer element is adapted to provide a first dampening effect on the lateral transition of the needle bar in a first direction, and the second buffer element is adapted to provide a second dampening effect on the lateral transition of the needle bar in a second direction.

Optionally, the tattoo machine assembly further comprises a third buffer element, said third buffer element being interchangeable with said first or second buffer element and being adapted to provide a third dampening effect on the lateral transition of the needle bar.

Optionally, the tattoo machine assembly further comprises a fourth buffer element, said fourth buffer element being interchangeable with said first or second buffer element and being adapted to provide a fourth dampening effect on the lateral transition of the needle bar.

Optionally, the tattoo machine assembly further comprises a fifth buffer element, said fifth buffer element being interchangeable with said first or second buffer element and being adapted to provide a fifth dampening effect on the lateral transition of the needle bar.

Optionally, the tattoo machine assembly further comprises a sixth buffer element, said sixth buffer element being interchangeable with said first or second buffer element and being adapted to provide a sixth dampening effect on the lateral transition of the needle bar.

In accordance with a fifth aspect, there is provided a kit comprising:
a tattoo machine;
at least one tattoo needle;
at least one tattoo needle tube; and
two or more buffer elements;
   the tattoo machine comprising:
a needle bar configured to translate in a lateral direction;
a motor adapted to actuate said needle bar in a lateral direction;
a tattoo needle tube holder adapted to hold said at least one tattoo needle tube; and
a housing configured to receive the needle bar and two buffer elements therein;
   wherein each buffer element is adapted to, in use, at last partially surround a portion of the needle bar and is adapted to provide a dampening effect on the lateral transition of the needle bar.

Optionally, at least one of the two or more buffer elements is adapted to provide a different dampening effect on the lateral transition of the needle bar to another of the two or more buffer elements.

Optionally, each of the two or more buffer elements is adapted to provide a different dampening effect on the lateral transition of the needle bar to another of the two or more buffer elements.

Optionally, the kit comprises six buffer elements.

Optionally, the six buffer elements are color coded in order to facilitate the identification of the amount of damping provided by the buffer element.

### BRIEF DESCRIPTION OF FIGURES

The invention will now be described by way of non-limiting example, with reference being made to the accompanying drawings, in which:

FIG. 1 depicts an embodiment of a tattoo machine in accordance with an aspect of the invention;

FIGS. 2a to 2c depict an embodiment of a buffer element forming part of a tattoo machine needle bar holder in accordance with an aspect of the invention;

FIG. 3 shows an embodiment of the machine needle bar holder in accordance with the invention;

FIG. 4 shows another embodiment of a machine needle bar holder in accordance with the invention;

FIG. 5 shows yet another embodiment of a machine needle bar holder in accordance with the invention; and

FIG. 6 depicts an embodiment of a tattoo machine assembly in accordance with an aspect of the invention incorporating the tattoo machine of FIG. 1.

### DETAILED DESCRIPTION

The preceding discussion of the background to the invention is intended only to facilitate an understanding of the present invention. It should be appreciated that the discussion is not an acknowledgement or admission that any of the material referred to was part of the common general knowledge as at the priority date of the application.

Throughout the description and claims of this specification, the words “comprise” and “contain” and variations of the words, for example “comprising” and “comprises”, means “including but not limited to”, and is not intended to (and does not) exclude other components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers or characteristics, compounds described in conjunction with a particular aspect, embodiment or example of the invention are to be understood to be applicable to any other aspect, embodiment or example described herein unless incompatible therewith.

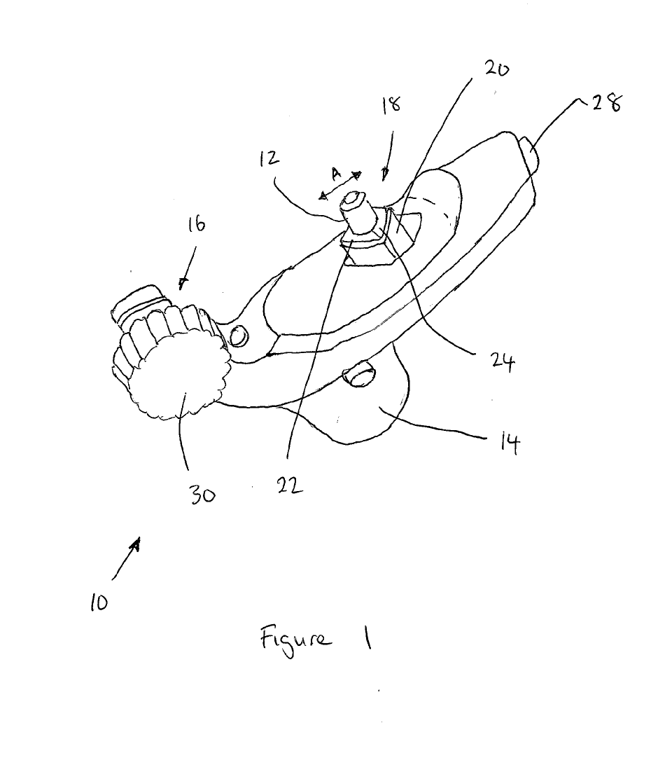

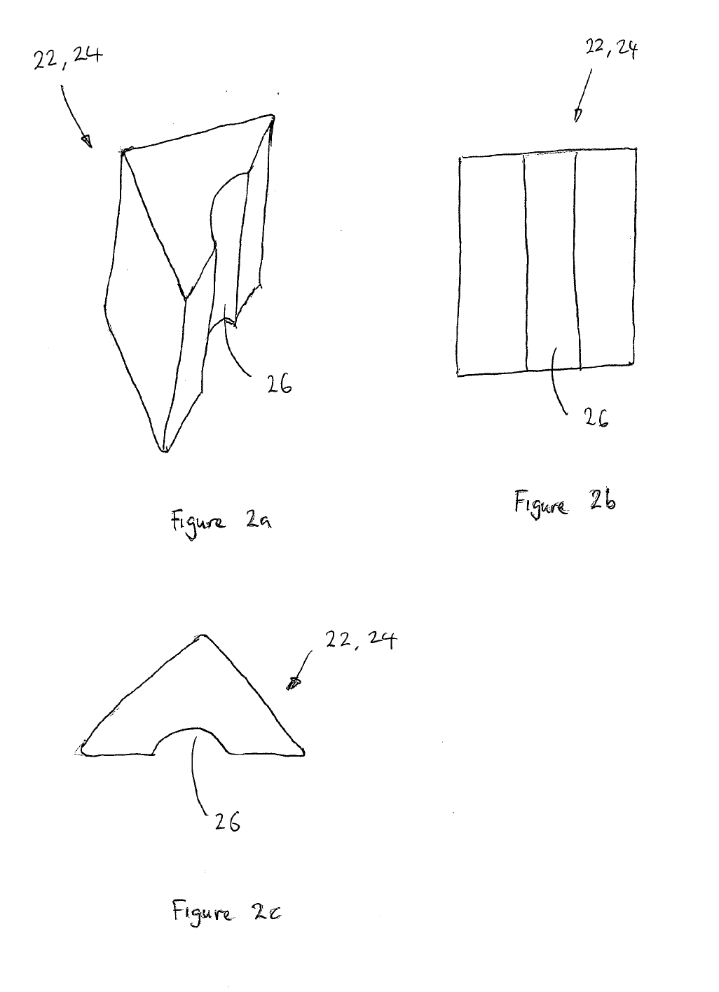

Referring to FIGS. 1 to 3, an embodiment of a tattoo machine 10 in accordance with an aspect of the invention is shown.

The tattoo machine 10 comprises needle bar 12 configured to translate in a lateral direction (as indicated by arrow A), a motor adapted to actuate the needle bar 12 in a lateral direction, a tattoo needle tube holder 16, and tattoo machine needle bar holder 18.

The tattoo machine 10 further comprises a power connector 28 operably connected to the motor 14 through which power can be supplied to the motor 14. In the embodiment shown, the power connector 28 is in the form of an RCA power connector although it would be understood that the power connector may take a different form.

The tattoo needle tube holder 16 is in the form of a vice/clamp-like structure and comprises an adjustable knob 30 for tightening or loosening the grip on the tattoo needle tube placed in the tattoo needle tube holder 16 as known in the art.

The tattoo machine needle bar holder 18 comprises a housing 20 configured to receive the needle 12 bar therein, a first buffer element 22, and a second buffer element 24.

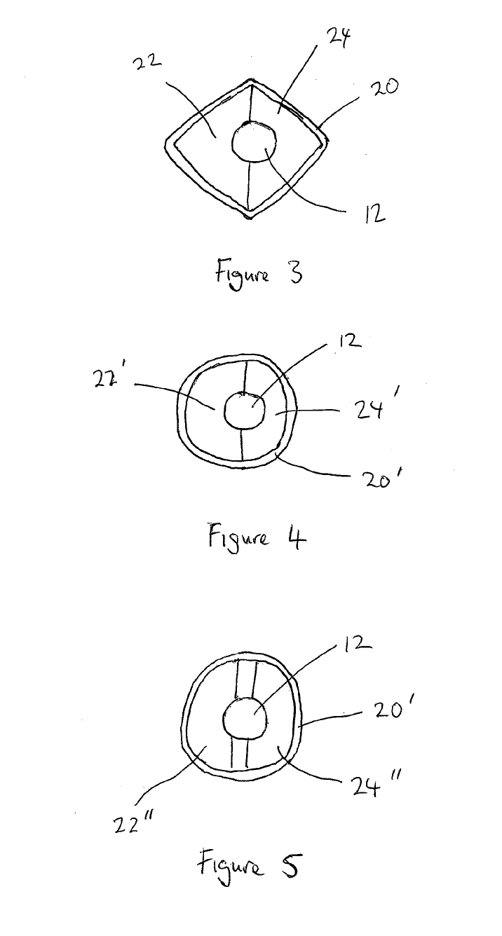

The first and second buffer elements 22, 24 are adapted to be located within the housing 20, and in use, at least partially surround a portion of a needle bar 12 located within said housing 20 (see FIGS. 1 and 3).

The first buffer element 22 is adapted to provide a first dampening effect on the lateral transition of the needle bar 12 located within said housing 20 in a first direction, and the second buffer element 24 is adapted to provide a second dampening effect on the lateral transition of the needle bar 12 in a second direction.

In the embodiment shown, the needle bar 12 is adapted to translate in a forward and back direction and as such, the first direction is opposite the second direction.

In the embodiment shown, the housing 20 is in the form of a hollow prism having a rhombus cross-sectional profile. Each buffer element 22, 24 is shaped to snugly fit within the housing 20 in use and comprises a recessed section 26 along a side thereof to accommodate a portion of the needle bar 12.

In the embodiment shown, the first and second buffer elements 22, 24 comprise a resilient material in the form of a rubber material. It would be understood that the buffer elements 22, 24 may comprise any suitable resilient material.

The rubber material for the first and second buffer elements 22, 25 preferably comprises a shore hardness equivalent to an international rubber hardness degrees (IRHD) value of between 10 and 100.

The first and second buffer elements 22, 24 are removably locatable in the housing 20 and their position within the housing may be interchanged.

Depending on the intended use of the tattoo machine, the first and second buffer elements 22, 24 can be selected such that the first dampening effect is the same as the second damping effect.

Alternatively, the first and second buffer elements 22, 24 may be selected such that the first dampening effect is different to the second damping effect. For example, the first and second buffer elements 22, 24 may be selected such that the first dampening effect is greater than the second damping effect or the first and second buffer elements 22, 24 may be selected such that the first dampening effect is less than the second dampening effect.

In this way, a tattoo artist can change the buffer elements 22, 24 in order to control the hardness and softness of the tattoo machine 10. In addition, by changing the damping effects on the lateral transition of the needle bar 12, the tattoo artist can use the same machine for both lining and shading.

While the housing 20 has been described in the form of a hollow prism having a rhombus cross-sectional profile. It would be understood that the housing may be any suitable shape.

For example, as shown in FIG. 4, the housing 20′ may be in the form of a hollow cylinder having circular cross-sectional profile. The shape of the first and second buffer elements 22′, 24′ will be such that they snugly fit within the housing 20′.

In addition, it is not necessary for the first and second buffer elements to completely surround a portion of a needle bar 12. FIG. 5 shows another embodiment of the first and second buffer elements 22″, 24″ falling within the scope of the invention. In this embodiment, the first and second buffer elements 22″, 24″ only partially surround a portion of the needle bar 12.

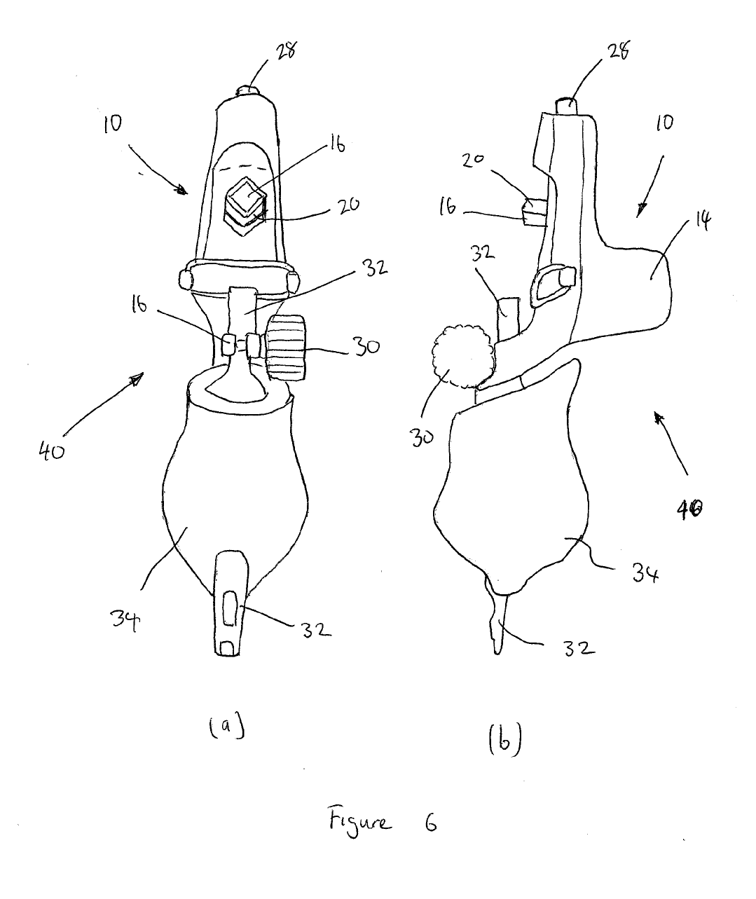

Referring to FIG. 6, an embodiment of a tattoo machine assembly 40 in accordance with an aspect of the invention is shown.

The tattoo machine assembly 40 comprises a tattoo machine as previously described (the needle bar 12 and the first and second buffer elements 22, 24 are not shown for clarity); and a tattoo needle tube 32.

The tattoo machine assembly 40 further comprises a grip 34 positioned over the tattoo needle tube 32.

In an embodiment of the invention, the tattoo machine assembly comprises six buffer elements, each adapted to provide a different dampening effect on the lateral transition of the needle bar to each other.

The buffer elements are each color coded in order to facilitate the identification of the amount of dampening provided by said buffer element. In an exemplary embodiment, the six buffer elements are color coded in the following order black-blue-red-purple-green-yellow, with a black colored buffer element providing the lowest dampening effect and a yellow colored buffer element providing the greatest dampening effect. The hardness value of each buffer element is chosen such that there is a 10% difference between adjacent colors. For example, a black colored buffer element would have a 10% lower hardness value than a blue colored buffer element, a blue colored buffer element would have a 10% lower hardness value than a red colored buffer element etc.

It would be understood that the tattoo machine assembly may comprise less or more that six buffer elements and that is it not necessary for all the buffer elements to provide a different dampening effect to each of the other dampening elements. Only of the plurality of buffer elements may provide a different dampening effect to the others.

The tattoo machine assembly 40 may form part of a kit further comprising at least one tattoo needle and at least one tattoo needle tube.

The present invention has been described in relation to particular embodiments, which are intended in all aspects to be illustrative rather than restrictive. Alternative embodiments will become apparent to those skilled in the art to which the present invention pertains without departing from its scope.

From the foregoing, it will be seen that this invention is one well adapted to attain all the ends and objects set forth above, together with other advantages which are obvious and inherent to the devices, assembly and kit. It will be understood that certain features and subcombinations are of utility and may be employed without reference to other features and subcombinations. This is contemplated and within the scope of the claims.

1. A tattoo machine needle bar holder comprising
a housing configured to receive a needle bar therein;
a first buffer element; and
a second buffer element;
wherein the first and second buffer elements are adapted to be located within said housing, and in use, at least partially surround a portion of a needle bar located within said housing,
wherein the first buffer element is adapted to provide a first dampening effect on the lateral transition of a needle bar located within said housing in a first direction, and the second buffer element is adapted to provide a second dampening effect on the lateral transition of the needle bar in a second direction.

2. The tattoo machine needle bar holder according to claim 1 wherein the first buffer element comprises a resilient material.

3. The tattoo machine needle bar holder according to claim 2 wherein the resilient material comprises a rubber material.

4. The tattoo machine needle bar holder according to claim 3 wherein the rubber material comprises an IRHD hardness value of between 10 and 100.

5. The tattoo machine needle bar holder according to claim 1 wherein the second buffer element comprises a resilient material.

6. The tattoo machine needle bar holder according to claim 5 wherein the resilient material comprises a rubber material.

7. The tattoo machine needle bar holder according to claim 6 wherein the rubber material comprises an IRHD hardness value of between 10 and 100.

8. The tattoo machine needle bar holder according to claim 1 wherein the first buffer element is removably locatable in the housing.

9. The tattoo machine needle bar holder according to claim 1 wherein the second buffer element is removably locatable in the housing.

10. The tattoo machine needle bar holder according to claim 1 wherein the first dampening effect is the same as the second dampening effect.

11. The tattoo machine needle bar holder according to claim 1 wherein the first dampening effect is different to the second dampening effect.

12. The tattoo machine needle bar holder according to claim 1 wherein the first dampening effect is different to the second dampening effect.

13. The tattoo machine needle bar holder according to claim 12 wherein the first dampening effect is greater than the second dampening effect.

14. The tattoo machine needle bar holder according to claim 12 wherein the first dampening effect is less than the second dampening effect.

15. The tattoo machine needle bar holder according to claim 1 wherein the first direction is opposite the second direction.

16. A tattoo machine comprising:
a needle bar configured to translate in a lateral direction;
a motor adapted to actuate said needle bar in a lateral direction;
a tattoo needle tube holder; and
a housing configured to receive the needle bar therein;
a first buffer element; and
a second buffer element;
wherein the first and second buffer elements are adapted to be located within said housing, and in use, at least partially surround a portion of the needle bar,
wherein the first buffer element is adapted to provide a first dampening effect on the lateral transition of the needle bar in a first direction, and the second buffer element is adapted to provide a second dampening effect on the lateral transition of the needle bar in a second direction.

17. A tattoo machine assembly comprising:
a needle bar configured to translate in a lateral direction;
a motor adapted to actuate said needle bar in a lateral direction;
a tattoo needle tube holder;
a tattoo needle tube;
a housing configured to receive the needle bar therein;
a first buffer element; and
a second buffer element;
wherein the first and second buffer elements are adapted to be located within said housing, and in use, at least partially surround a portion of the needle bar,
wherein the first buffer element is adapted to provide a first dampening effect on the lateral transition of the needle bar in a first direction, and the second buffer element is adapted to provide a second dampening effect on the lateral transition of the needle bar in a second direction.

18. The tattoo machine assembly according to claim 17 further comprising a third buffer element, said third buffer element being interchangeable with said first or second buffer element and being adapted to provide a third dampening effect on the lateral transition of the needle bar.

19. The tattoo machine assembly according to claim 18 further comprising a fourth buffer element, said fourth buffer element being interchangeable with said first or second buffer element and being adapted to provide a fourth dampening effect on the lateral transition of the needle bar.

20. The tattoo machine assembly according to claim 19 further comprising a fifth buffer element, said fifth buffer element being interchangeable with said first or second buffer element and being adapted to provide a fifth dampening effect on the lateral transition of the needle bar.

21. The tattoo machine assembly according to claim 20 further comprising a sixth buffer element, said sixth buffer element being interchangeable with said first or second buffer element and being adapted to provide a sixth dampening effect on the lateral transition of the needle bar.

22. A kit comprising:
a tattoo machine;
at least one tattoo needle;
at least one tattoo needle tube; and
two or more buffer elements;
the tattoo machine comprising:
a needle bar configured to translate in a lateral direction;
a motor adapted to actuate said needle bar in a lateral direction;
a tattoo needle tube holder adapted to hold said at least one tattoo needle tube; and
a housing configured to receive the needle bar and two buffer elements therein;
wherein each buffer element is adapted to, in use, at least partially surround a portion of the needle bar and is adapted to provide a dampening effect on the lateral transition of the needle bar.

23. The kit according to claim 22 wherein at least one of the two or more buffer elements is adapted to provide a different dampening effect on the lateral transition of the needle bar to another of the two or more buffer elements.

24. The kit according to claim 22 wherein each of the two or more buffer elements is adapted to provide a different dampening effect on the lateral transition of the needle bar to another of the two or more buffer elements.

23. The kit according to claim 22 comprising six buffer elements.

24. The kit according to claim 23 wherein each buffer element is color coded.
